(19) United States
(12) Patent Application Publication
(10) Pub. No.: US 2005/0222538 A1
(43) Pub. Date: Oct. 6, 2005

(54) Surgical system for delivery of viscous fluids

(75) Inventors: Jill Embry, Memphis, TN (US); William Albans, Cordova, TN (US); Troy D. Dewry, Memphis, TN (US); Andrew M. Dickson, Memphis, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 10/812,582

(22) Filed: Mar. 30, 2004

(51) Int. Cl. A61M 5/00

(52) U.S. Cl. 604/181; 604/82

(57) ABSTRACT

A surgical system for delivery of viscous fluid is described. The system includes a housing having a handle portion and a coupling portion extending generally transverse to the handle portion, a reservoir member for engaging with the housing, the reservoir member being adapted to receive a viscous fluid prior to engagement with the housing, and a plunger member adapted to be inserted into the coupling portion and translated through the reservoir member via engagement of the plunger member with the coupling portion.

### BACKGROUND

The present disclosure relates generally to the field of orthopedics and spinal surgery, and in some embodiments, the present disclosure relates to a system for injecting a viscous fluid into a human body.

In the treatment of diseases, injuries or malformations affecting the skeletal support structure, it has long been known to remove or supplement some or all of a degenerated, ruptured or otherwise failing skeletal support structure. For example, compressive loads experienced due to accidents can result in vertebral compression fractures, which can lead to further complications. It is has been found that such compression fractures, and various other fracture trauma in general, can be treated with the application of a viscous fluid, such as polymethylmethacrylate (PMMA), to the fracture region. Further, diseased bone tissue subject to compression or fracture may be supplemented with viscous graft material promoting the formation of stronger or more dense bone.

Therefore, what is needed is a viscous fluid delivery system for the insertion of a viscous fluid into an implant site.

### SUMMARY

A surgical system for delivery of viscous fluid is described. The system includes a housing having a handle portion and a coupling portion extending generally transverse to the handle portion, a reservoir member for engaging with the housing, the reservoir member being adapted to receive a viscous fluid prior to engagement with the housing, and a plunger member adapted to be inserted into the coupling portion and translated through the reservoir member via engagement of the plunger member with the coupling portion.

A surgical system for delivery of viscous fluid is described. The system includes a reservoir member for storing a viscous fluid, the reservoir member having an orifice defined in an end thereof, a plunger member adapted to engage the reservoir member in a threaded engagement, and a mixer assembly extending through the plunger member, the mixer assembly being rotatable to mix the viscous fluid, wherein the plunger member is adapted to be translated through the reservoir member to eject the viscous fluid through the orifice of the reservoir member.

A surgical system for delivery of viscous fluid is described. The system includes means for storing a viscous fluid, means for ejecting the viscous fluid from the means for storing, and means for mixing the viscous fluid within the means for storing, the means for mixing being movable within the means for ejecting.

A surgical system for delivery of viscous fluid is described. The system includes a housing having a coupling portion and a handle portion extending obliquely from the coupling portion, a reservoir member having a threaded portion to engage with a threaded annular flange defined at a first end of the housing, the reservoir member being adapted to store viscous material therein, a plunger member insertable into a second end of the housing opposing the first end of the housing, the plunger member having a threaded member to engage a threaded annular surface of the coupling portion, and a plunger head disposed on an end of the plunger member, the plunger head being adapted to drive and eject viscous fluid from the reservoir member via actuation of the plunger member.

A surgical method for delivering viscous fluid to an implant site is described. The method includes providing a modular delivery system, the delivery system having a housing, a plunger member engageable with a first end of the housing, and a reservoir member engageable with a second end of the housing. The method further includes loading viscous fluid into the reservoir member, engaging the reservoir member with the housing, engaging the plunger member with the housing, and actuating the plunger member through the housing and into the reservoir member to eject viscous fluid from the reservoir member.

A surgical method for delivering implant material to an implant site is described. The method includes providing a plunger member having a bore defined therethrough, disposing a mixer assembly through the bore of the plunger member, inserting the plunger member into a reservoir member, the reservoir member having viscous fluid therein, actuating the mixer assembly to mix the viscous fluid in the reservoir member, and actuating the plunger member to eject viscous fluid from the reservoir member.

### BRIEF DESCRIPTION OF THE DRAWINGS

FIG. 1 is a perspective view of a viscous fluid delivery system according to one embodiment of the present disclosure.

FIG. 2 is a sectional view of the viscous fluid delivery system of FIG. 1 depicting a plunger head associated with the viscous fluid delivery system in a first position.

FIG. 3 is an exploded view of the sectioned components of FIG. 2.

FIG. 4 is a detailed view of a tube attachment associated with the viscous fluid delivery system.

FIG. 5 is a perspective view of the viscous fluid delivery system of FIG. 1 positioned adjacent to an implant site.

FIG. 6 is a perspective view of a viscous fluid delivery system according to another embodiment of the present disclosure.

FIG. 7 is a sectional view of a reservoir and a perspective view of a funnel each being associated with the viscous fluid delivery system of FIG. 6.

FIG. 8 is part sectional, part perspective view of the viscous fluid delivery system of FIG. 6 shown with a mixer assembly associated therewith.

FIG. 9 is an exploded view of the mixer assembly depicted in FIG. 8 with a portion of the plunger broken away.

### DESCRIPTION

For the purposes of promoting an understanding of the principles of the disclosure, reference will now be made to the embodiments, or examples, illustrated in the drawings and specific language will be used to describe the same. It will nevertheless be understood that no limitation of the scope of the disclosure is thereby intended. Any alterations and further modifications in the described embodiments, and any further applications of the principles of the disclosure as described herein are contemplated as would normally occur to one skilled in the art to which this disclosure relates.

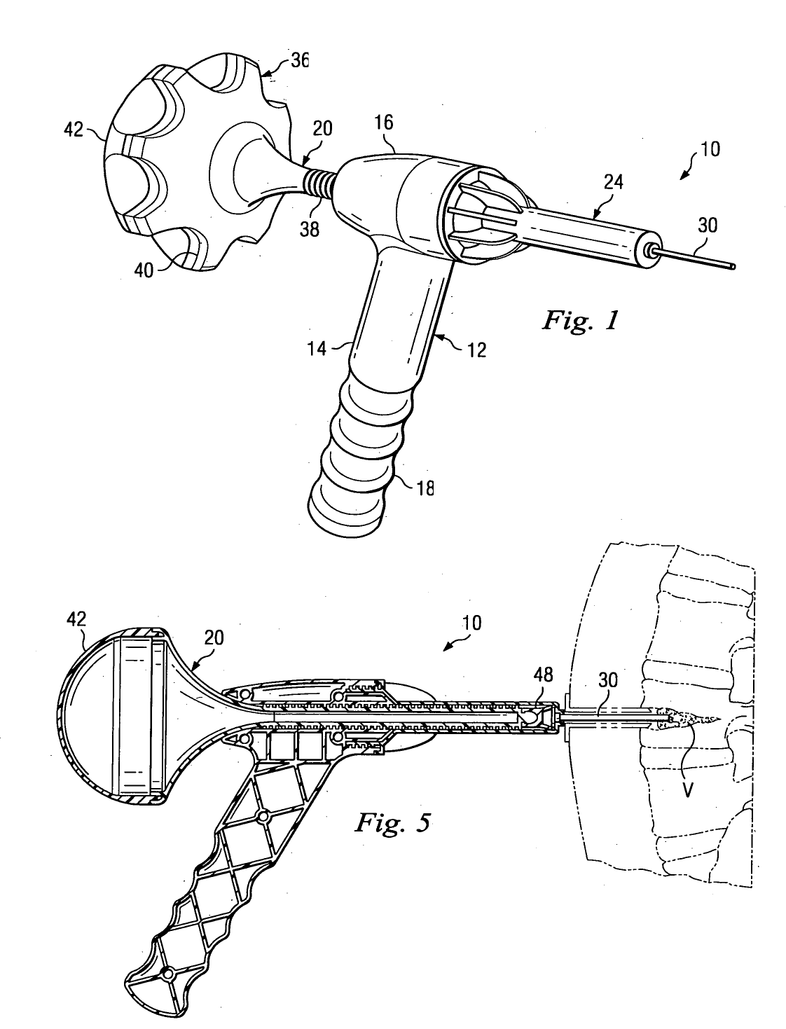

Referring now to FIGS. 1-3, a viscous fluid delivery system for delivering a viscous fluid to an implant site is generally referred to by reference numeral 10. In one embodiment, the viscous fluid delivery system 10 includes a housing 12 having a handle portion 14 and a coupling portion 16 extending generally transverse to the handle portion. In the present example, the handle portion 14 is depicted as having an undulating outer surface 18 to aid in gripping the handle portion. However, it is understood that a variety of surface configurations are contemplated for the handle portion 14. In some embodiments, the handle portion 14 may be hollow to lessen the weight of the system 10 and to reduce the costs associated with manufacturing the system.

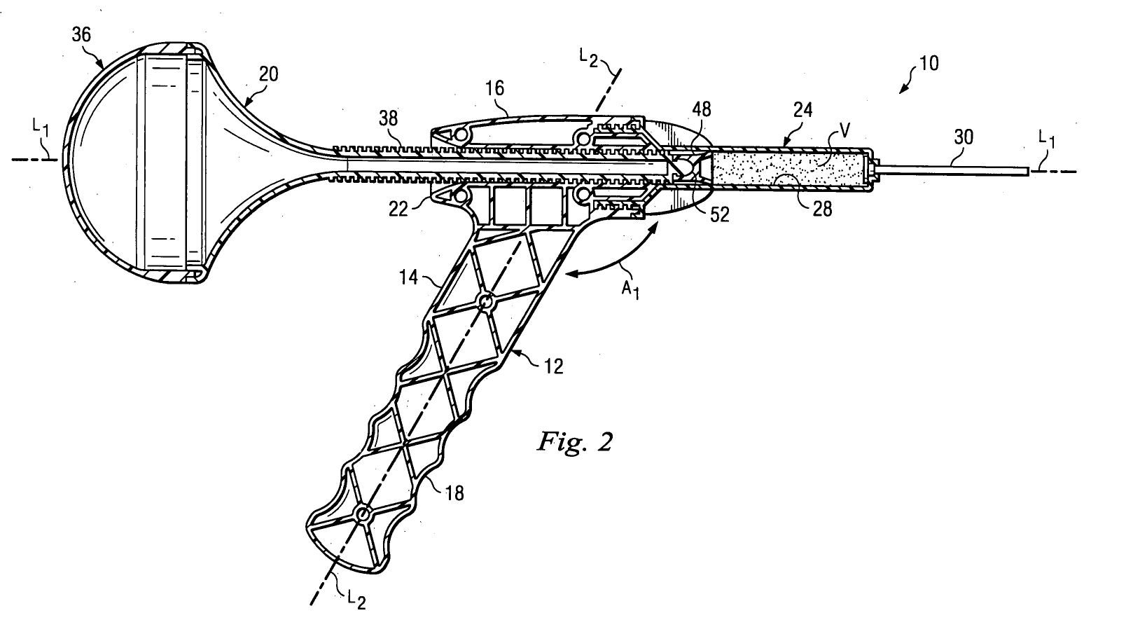

The delivery system 10 further includes a plunger member 20, which is adapted to be inserted into a proximal end 22 of the housing 12. Also provided with the delivery system 10 is a reservoir member 24, which secures to a distal end 26 of the coupling portion 16 and is adapted to receive and store a viscous fluid, generally depicted as V in FIG. 2, in an annular cavity 28 thereof as will be described. In one embodiment, the viscous fluid V is a bone-void filling substance such as bone cement, polymethylmethacrylate (PMMA), calcium phosphate (CaP), demineralized bone matrix (DBM), bi-calcium phosphate matrix, platelet gel, autograft, allograft, bone morphogenetic protein (BMP) in a carrier matrix, or one or more of the previous in combination. Furthermore, it is understood that for purposes of this disclosure, the term “proximal” refers to the direction generally towards a user (not shown) of the viscous fluid delivery system 20 such as a surgeon, and the term “distal” refers to the direction generally towards a patient (not shown). The delivery system 10 further includes a tubing 30, which is adapted to secure to and through an orifice 32 defined in a distal end 34 of the reservoir member 24 for reasons also to be described.

The plunger member 20 includes a knob 36 and a threaded member 38 integrally formed with the knob. In one embodiment, the knob 36 includes an undulating gripping surface 40 along a perimeter, which aids in gripping of the knob. As is readily apparent from FIG. 1, the knob 32 also includes a generally uniform compression surface 42, which is adapted to receive a compressive force during actuation of the knob. In the illustrated embodiment, the compression surface 42 is hemispherical. Accordingly, the knob 32 is configured for simultaneous compression and rotation. It is understood that various other configurations of the knob 32 are contemplated such as configurations in which the knob is generally uniform along its entire surface.

Moreover, the arrangement of the handle portion 14 relative to the coupling portion 16, the plunger member 20 and the reservoir member 24 may be configured in a variety of ways to aid use of the system 10. For example, referring to FIG. 2, the handle portion 14 may extend from the coupling portion 16 in an angled, or oblique, manner such that an obtuse angle A1 defines the relationship between a longitudinal axis L₁ of the coupling portion and a longitudinal axis L₂ of the handle portion.

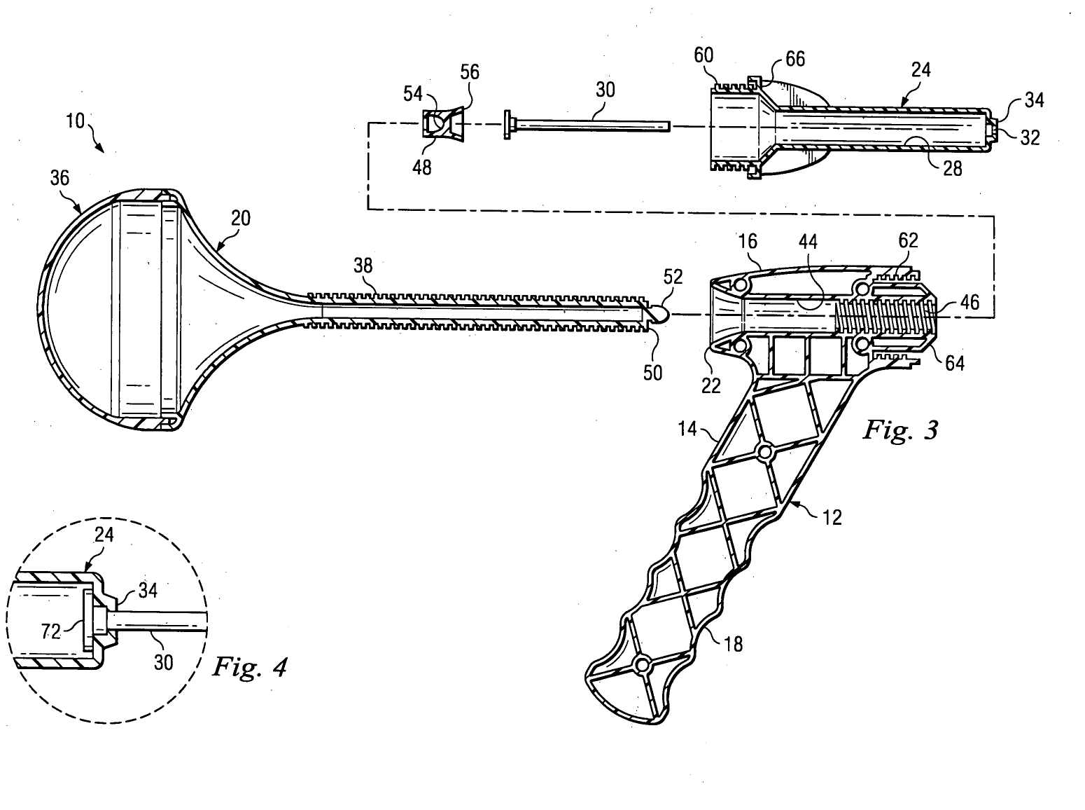

Referring to FIGS. 2-3, the threaded member 38 is adapted to be disposed within the coupling portion 16 of the housing 12. The coupling portion 16 includes an inner annular surface 44, which is threaded along a portion thereof to define a threaded surface 46 of the inner annular surface 44. As such, the threaded member 38 of the plunger member 20 can engage the threaded surface 46 of the coupling portion 16, which facilitates rotation and translation of the plunger member 20 through the coupling portion 16 and into the reservoir member 24.

In the present example, a plunger head 48 is press-fit onto a distal end 50 of the plunger member 20. This connection may allow rotation of the distal end 50 with respect to plunger head 48. To facilitate the press-fit connection, a flange member 52 extends from the distal end 50 to engage with a corresponding recess 54 defined in the plunger head 48. In one embodiment, the plunger head 48 is substantially flush with the distal end 50 of the plunger member 20 when engaged therewith. Moreover, a portion of the plunger head 48 is flared outward such that a distal end 56 of the plunger head 48 has a circumference that is substantially equal to the circumference of the annular cavity 28 of the reservoir member 24. Accordingly, translation of the plunger member 20 through the reservoir member 24 causes ejection of viscous fluid from the delivery system 10 as will be described. As can be appreciated, the relative larger diameter of the knob 36 as compared to the plunger head 48 aids in actuation of the delivery system 10.

The reservoir member 24 is adapted to engage with the coupling portion 16 of the housing 12 such that the annular cavity 28 retains the viscous fluid V in an impermeable manner. In one embodiment, the reservoir member 24 includes a threaded proximal end 60, which is adapted to secure to a corresponding threaded annular flange 62 of the coupling portion 16. The coupling portion 16 additionally includes an inner annular flange 64 relative to the threaded annular flange 62 such that the engagement of the reservoir member 24 with the coupling portion forms a sealed engagement. To further facilitate the sealed engagement, the reservoir member 24 includes an annular shoulder 66, which is adapted to abut against the threaded annular flange 62 of the coupling portion 16. The relative larger diameter of the annular flange 62 as compared to the diameter of the threaded surface 46 of the coupling portion 16 offers additional control in handling of the delivery system 10. Of course, a variety of sealing arrangements between the coupling portion 16 and the reservoir member 24 are contemplated other than the detailed sealing arrangement described above. For example, it is contemplated that the reservoir member 24 may be snap-fit to the housing 12.

The tubing 30 is inserted through the distal end 34 of the reservoir member 24 to provide a conduit through which to pass the viscous fluid V from the reservoir member to an implant site (not depicted), which may be any region of the human body for which delivery of the viscous fluid V is desired. For example, the viscous fluid may be used in repairing a fractured vertebral body, and as such, may be delivered to the fractured region of the vertebral body. However, the viscous fluid delivery system 10 may be used in the treatment of other spinal disorders, or in some embodiments, non-spinal disorders.

It is understood that the tubing 30 may be inserted through the distal end 34 of the reservoir member 24 in a variety of manners. For example, and referring to FIG. 4, the tubing 30 may be inserted into the reservoir member 24 in a press-fit manner such that a tubing flange 72 abuts against the reservoir member while the remaining portion of the tubing passes through the orifice 32 of the reservoir member. It is further understood that the tubing 30 may cooperate with another conduit, such as a cannula (not depicted), during passage of the viscous fluid V therethrough. In one example, the cannula may be previously placed adjacent to the implant site, and the tubing 30 may be inserted through the cannula until reaching the implant site.

The various components of the delivery system 10, such as the housing 12, the plunger member 20 and the reservoir member 24, may be manufactured from a variety of materials. For example, the delivery system 10 may be formed of disposable materials such as disposable plastic, or alternatively, the delivery system may be formed of materials which facilitate reuse of the delivery system.

Referring again to FIG. 1, in operation, the delivery system 10 is assembled by snap-fitting the plunger head 48 onto the plunger member 20. The plunger member 20 is then inserted into the proximal end 22 of the coupling portion 16 to engage the threaded member 38 with the threaded surface 46 of the housing 12. The tubing 30 is inserted through the reservoir member 24, and subsequently, viscous fluid V is loaded into the reservoir member. The reservoir member 24 is then engaged with the housing 12, thereby readying the delivery system 10 for use. It is understood that the above-described components of the delivery system 10 may be provided pre-assembled, or, in other embodiments, may be provided in modular form to be assembled by a user.

Referring to FIGS. 2 and 5, the delivery system 10 is positioned adjacent to an implant site in preparation of injection of the viscous fluid V therein. Forming and accessing the implant site may be accomplished in a variety of ways including via the methods disclosed in pending application Ser. No. 09/928,949 incorporated by relevance herein in its entirety. Upon proper positioning, the knob 36 is rotated, which translates the plunger head 48 against the viscous fluid V to force the viscous fluid into the tubing 30, and subsequently to the implant-site. As can be appreciated, the plunger member 20 can be translated through the housing to the point of contacting the plunger head 48 with the tubing 30 as is depicted in FIG. 5. Accordingly, a majority, if not all, of the viscous fluid V and other material loaded into the reservoir 24 may be delivered to the implant site. However, it is understood that any amount of the viscous fluid V initially deposited into the reservoir member 24 may be delivered to the implant site.

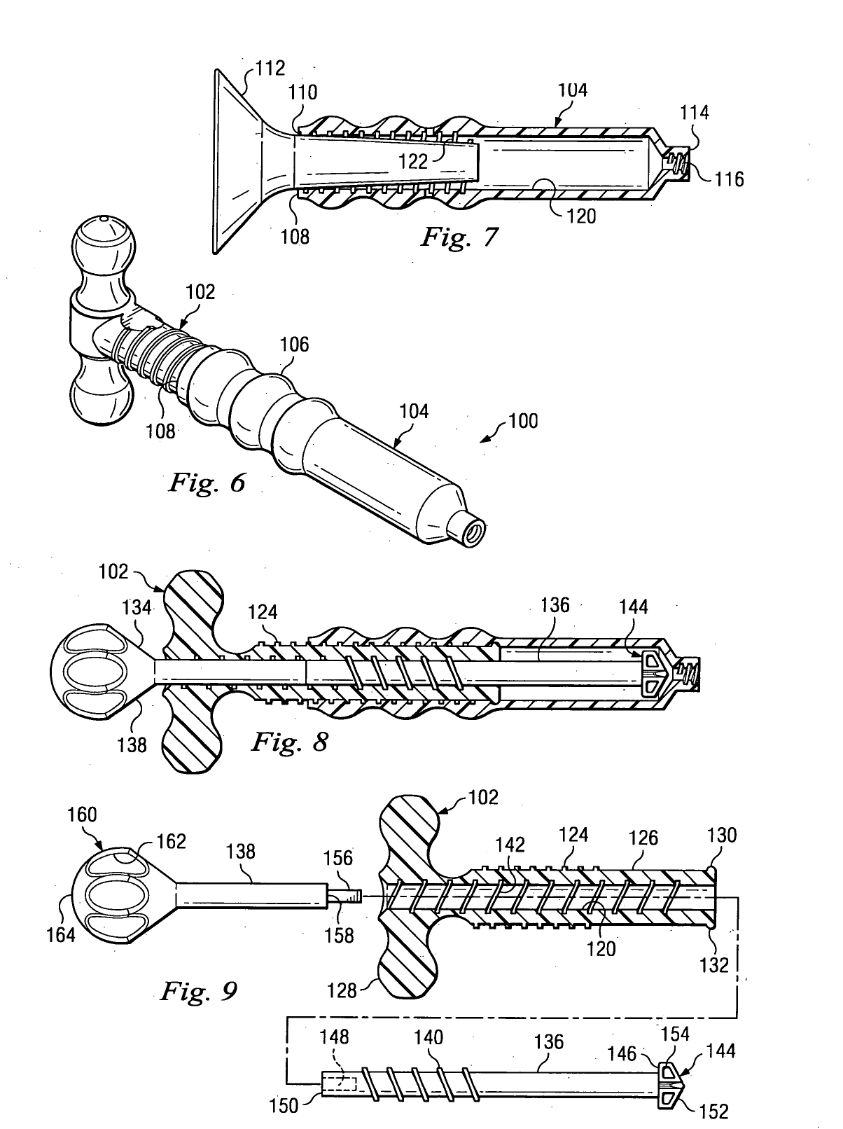

It is understood that variations may be made in the foregoing without departing from the scope of the disclosure. For example, and referring now to FIG. 6, in an alternative embodiment, a viscous fluid delivery system is generally referred to by reference numeral 100. The delivery system 100 includes a plunger member 102, which is disposed and movable within a reservoir member 104. The plunger member 102 is adapted to eject viscous fluid from the reservoir member 104 in a manner to be described. The reservoir member 104 is shaped to facilitate gripping thereof, and as such, in one embodiment, includes an undulating surface 106 adjacent to a proximal end 108 of the reservoir member.

Referring to FIG. 7, the reservoir member 104 is generally hollow and includes an opening 110 at the proximal end 108 such that various instruments may be used with the reservoir member other than the plunger member 102 (FIG. 6). For example, a funnel 112 may be used with the reservoir member 104 to facilitate loading of viscous fluid (not shown) into the reservoir member. A distal end 114 of the reservoir member 104 is reduced in diameter relative to the rest of the reservoir member, and includes an orifice 116 defined therethrough for allowing for the passage of viscous fluid. The orifice 116 may be partially threaded to receive various attachments such as a plug (not shown) to contain viscous fluid within the reservoir member 104, or, should passage of viscous fluid through the orifice be desired, the threaded member of the orifice may receive a tubing attachment (not shown) or a coupling mechanism for coupling the reservoir member to a tubing or the like. Of course, the orifice 116 may receive such attachments in ways other than via a threaded connection, such as snap-fit connections.

Referring to FIGS. 7-9, the reservoir member 104 includes an inner annular surface 120, a portion of which is formed as a threaded receiving surface 122 for engaging with a corresponding threaded surface 124 associated with the plunger member 102. Accordingly, the plunger member 102 can be actuated to translate and rotate through the reservoir member 104 as will be described. Referring to FIGS. 8 and 9, the plunger member 102 is generally T-like in shape to include an annular portion 126 and a transverse handle portion 128. The annular portion 126 includes a gasket 130 on a distal end 132 thereof. In one embodiment, the gasket 130 is a flared-out portion of the plunger member 102 that is substantially similar in diameter to the diameter of the inner annular surface 120 of the reservoir member 104.

A bore 132 is defined through the plunger member 102 to receive a mixer assembly 134, which includes a mixer 136 and a mixer handle 138. The bore 132 may be configured for substantially fluid-tight engagement with the mixer 136. Although not shown, one or more seals may be disposed between mixer 136 and bore 132. The mixer 134 includes a threaded surface 140 along a portion thereof to engage with a threaded inner annular surface 142 of the plunger member 102. The mixer 136 further includes a mixer head 144 disposed at a distal end 146 of the mixer and a receptacle 148 formed in a proximal end 150 of the mixer. The mixer head 144 may be configured in any manner to facilitate mixing of the viscous fluid within the reservoir member 104. For example, as depicted, the mixer head 144 is formed as a plurality of mixing blades 152 having slots 154 defined therethrough.

Moreover, the plunger member 102 is adapted to receive the mixer handle 138 therein via the bore 132. The mixer handle 138 includes a post 156 extending from a distal end 158 of the mixer handle such that the mixer handle can engage the mixer 134 via the post and the corresponding receptacle 148 defined in the mixer. A knob 160, associated with the mixer handle 136, includes an undulating gripping surface 162, which aids in gripping of the knob. The knob 160 also includes a generally uniform compression surface 164, which is adapted to receive a compressive force during actuation of the knob. Accordingly, the knob 156 is configured for simultaneous compression and rotation. As such, compression and rotation of the knob 160 imparts translation and rotation to the mixer 136 and the associated mixer head 144. It is understood that various other configurations of the knob 160 are contemplated such as configurations in which the knob is generally uniform along its entire surface.

In operation, viscous fluid is loaded into the reservoir member 104 via the funnel 112. The mixer 136 is then threaded into the plunger member 102 and the mixer handle 138 is engaged with the mixer through the plunger member. In addition or alternatively, plunger member 102 may be moved to draw liquids such as blood, platelet gel or saline into the reservoir member 104 for mixing with other materials. The mixer handle 138 is then actuated to impart rotation to the mixer head 144, thereby effectively mixing the materials disposed in the reservoir member 104. After mixing the materials, the viscous fluid is then ejected from the delivery system 100 by translating the plunger member 102 through the reservoir member 104 via rotation of the plunger member. Although not shown, it is understood that a tubing or some other conduit may be attached to the delivery system 100 to aid in delivery of the materials to the intended implant site (not shown). Also, to aid in rotation of the plunger member 102, the mixer handle 138 may be removed from the delivery system 100 prior to actuation of the plunger member 102.

The present disclosure has been described relative to several preferred embodiments. Improvements or modifications that become apparent to persons of ordinary skill in the art after reading this disclosure are deemed within the spirit and scope of the application. For example, although described with respect to a particular shape, it is understood that the housing 12 may be take a variety of orientations to facilitate the deposit of viscous fluid V and/or other materials from the delivery system 10 to the implant site. Moreover, a variety of tubing devices may be used with either of the viscous fluid delivery systems 20 and 100. For instance, a compliant tubing may be used in conjunction with a non-compliant tubing sheath to pass viscous fluid from the delivery systems 20, 100 to a desired implant site.

Accordingly, it is understood that several modifications, changes and substitutions are intended in the foregoing disclosure and, in some instances, some features of the disclosure will be employed without a corresponding use of other features. It is also understood that all spatial references, such as “proximal,” “distal,” and “transverse,” are for illustrative purposes only and can be varied within the scope of the disclosure. Accordingly, it is appropriate that the appended claims be construed broadly and in a manner consistent with the scope of the disclosure.

1. A surgical system for delivery of viscous fluid, comprising a housing having a handle portion and a coupling portion extending generally transverse to the handle portion, a reservoir member for engaging with the housing, the reservoir member being adapted to receive a viscous fluid prior to engagement with the housing, and a plunger member adapted to be inserted into the coupling portion and translated through the reservoir member via engagement of the plunger member with the coupling portion.

2. The system of claim 1 wherein the plunger member comprises a knob and a threaded member integrally formed with the knob.

3. The system of claim 2 wherein a portion of the knob is generally hemispherical, and a portion of the knob is generally undulating in cross-section.

4. The system of claim 3 wherein the portion of the knob that is generally hemispherical is a compression surface.

5. The system of claim 2 wherein the threaded member is adapted to engage a threaded region formed on an inner surface of the coupling portion.

6. The system of claim 1 further comprising an orifice defined through a distal end of the reservoir member.

7. The system of claim 6 further comprising a tubing inserted through the reservoir member via the orifice.

8. The system of claim 1 wherein the handle portion of the housing comprises an undulating surface.

9. The system of claim 2 wherein the plunger member further comprises a plunger head disposed on the plunger member, the plunger head being adapted to engage and drive the viscous fluid through the reservoir.

10. The system of claim 9 wherein the plunger head is flared outward to have a circumference of substantially equal size as the circumference of an inner annular surface of the reservoir.

11. The system of claim 1 wherein the distal end of the housing comprises a threaded annular flange for receiving a corresponding threaded proximal end of the reservoir member in a sealed engagement.

12. The system of claim 1 wherein the handle portion extends obliquely from the coupling portion to define an obtuse angle between the handle portion and the coupling portion.

13. The system of claim 9 wherein the diameter of the knob is larger than the diameter of the plunger head.

14. A surgical system for delivery of viscous fluid, comprising a reservoir member for storing a viscous fluid, the reservoir member having an orifice defined in an end thereof, a plunger member adapted to engage the reservoir member in a threaded engagement, and a mixer assembly extending through the plunger member, the mixer assembly being rotatable to mix the viscous fluid, wherein the plunger member is adapted to be translated through the reservoir member to eject the viscous fluid through the orifice of the reservoir member.

15. The system of claim 14 wherein the reservoir member further comprises an opening formed in an end of the reservoir member opposing the orifice end, the opening being adapted to receive the plunger member.

16. The system of claim 15 further comprising a funnel adapted to be inserted into the opening for aiding in the delivery of viscous fluid into the reservoir member.

17. The system of claim 14 wherein the plunger member is generally T-like in shape.

18. The system of claim 14 wherein the mixer assembly comprises a mixer and a mixer handle connectable with the mixer.

19. The system of claim 18 wherein the mixer includes a receptacle defined therein for receiving a corresponding post extending from the mixer handle.

20. The system of claim 18 wherein the mixer is adapted to be inserted into a first end of the plunger member and the mixer handle is adapted to be inserted into a second end of the plunger member, the second end opposing the first end.

21. The system of claim 18 wherein the mixer comprises a mixer head disposed on an end of the mixer, the mixer bead having a plurality of mixer blades.

22. The system of claim 21 wherein the mixer blades each comprise a slot defined therethrough.

23. The system of claim 18 wherein the plunger member includes a bore defined therethrough, the bore defining an annular surface of the plunger member.

24. The system of claim 23 wherein the annular surface of the plunger member is threaded along a portion thereof.

25. The system of claim 24 wherein the mixer includes a threaded surface for engaging with the threaded portion of the inner annular surface of the plunger member.

26. The system of claim 14 wherein the reservoir member includes an undulating surface along a portion thereof.

27. A surgical system for delivery of viscous fluid, comprising means for storing a viscous fluid, means for ejecting the viscous fluid from the means for storing, and means for mixing the viscous fluid within the means for storing, the means for mixing being movable within the means for ejecting.

28. A surgical system for delivery of viscous fluid, comprising:
a housing having a coupling portion and a handle portion extending obliquely from the coupling portion;
a reservoir member having a threaded portion to engage with a threaded annular flange defined at a first end of the housing, the reservoir member being adapted to store viscous material therein;
a plunger member insertable into a second end of the housing opposing the first end of the housing, the plunger member having a threaded member to engage a threaded annular surface of the coupling portion; and
a plunger head disposed on an end of the plunger member, the plunger head being adapted to drive and eject viscous fluid from the reservoir member via actuation of the plunger member.

29. The system of claim 28 wherein the plunger member further comprises a knob wherein a portion of the knob includes a generally hemispherical compression surface, and a portion of the knob includes a generally undulating surface.

30. A surgical method for delivering viscous fluid to an implant site, comprising:
providing a modular delivery system, the delivery system having a housing, a plunger member engageable with a first end of the housing, and a reservoir member engageable with a second end of the housing;
loading viscous fluid into the reservoir member;
engaging the reservoir member with the housing;
engaging the plunger member with the housing; and
actuating the plunger member through the housing and into the reservoir member to eject viscous fluid from the reservoir member.

31. The method of claim 30 wherein engaging the reservoir member with the housing comprises engaging a threaded end of the reservoir member with a threaded annular flange of the housing.

32. The method of claim 30 wherein engaging the plunger member with the housing comprises inserting the plunger member into the housing and engaging a threaded portion of the plunger member with a threaded annular surface of the housing.

33. The method of claim 30 wherein actuating the plunger member comprises applying a compressive force and a rotational force to a knob provided with the plunger member.

34. A surgical method for delivering implant material to an implant site, comprising:
providing a plunger member having a bore defined therethrough;
disposing a mixer assembly through the bore of the plunger member;
inserting the plunger member into a reservoir member, the reservoir member having viscous fluid therein;
actuating the mixer assembly to mix the viscous fluid in the reservoir member; and
actuating the plunger-member to eject viscous fluid from the reservoir member.

35. The method of claim 34 wherein the mixer assembly comprises a mixer and a mixer handle, the mixer being adapted to engage the plunger member in a threaded engagement through a first opening defined by the bore, and the mixer handle being adapted to be disposed through a second opening defined by the bore to connect with the mixer.

36. The method of claim 34 wherein inserting the plunger member into the reservoir member comprises engaging a threaded surface of the plunger member with a threaded annular surface of the reservoir member.

37. The method of claim 34 wherein actuating the mixer assembly comprises applying a compressive force and a rotational force to a knob provided with the mixer handle.
